(19) United States
(12) Patent Application Publication
(10) Pub. No.: US 2026/0309617 A1
(43) Pub. Date: Oct. 8, 2026

(54) CONCRETE STRUCTURE PROTECTION SYSTEM INTEGRATING DAMAGE SENSING AND CATHODIC PROTECTION

(71) Applicants: Zhejiang University of Science and Technology, Hangzhou City (CN); Zhejiang University of Technology, Hangzhou City (CN); Shenyang Jianzhu University, Shenyang City (CN); Ningxia University, Yinchuan City (CN); Shandong Transportation Institute, Jinan City (CN)

(72) Inventors: Xin ZHAO, angzhou City (CN); Jintao LIU, Hangzhou City (CN); Bing WANG, Shenyang City (CN); Ping WU, Yinchuan City (CN); Baolin GUO, Jinan City (CN); Shuai LIU, Jinan City (CN); Taihao ZHENG, Hangzhou City (CN)

(21) Appl. No.: 19/630,307

(22) Filed: Mar. 26, 2026

(30) Foreign Application Priority Data
Apr. 7, 2025 (CN) 202510426277.9

(51) Int. Cl. G01N 17/02; G01N 33/38

(52) U.S. Cl. CPC G01N 17/02; G01N 33/383

(57) ABSTRACT

A reinforced concrete dynamic protection system with both damage self-sensing and active corrosion protection is provided, including a rebar skeleton, conductive self-sensing concrete, a stainless steel electrode, a solar power supply, and a monitoring control system. The rebar skeleton is connected to a negative electrode of the solar power supply, and the conductive self-sensing concrete is poured around the rebar skeleton as a conductive path and crack monitoring; the stainless steel electrode is buried on a surface layer of the self-sensing concrete and is connected to an anode of the solar power supply to form a cathodic protection system loop; and the monitoring control system is configured to analyze an electrical signal in the system loop and control a frequency and voltage of a pulsed current.

### CROSS-REFERENCE TO RELATED APPLICATION

This patent application claims the benefit and priority of Chinese Patent Application No. 202510426277.9 filed with the China National Intellectual Property Administration on Apr. 7, 2025, the disclosure of which is incorporated by reference herein in its entirety as part of the present application.

### TECHNICAL FIELD

The present disclosure belongs to the technical field of rebar corrosion protection for reinforced concrete structures, and particularly relates to a cracking sensing and protection system based on self-sensing concrete.

### BACKGROUND

In the prior art, such as a corrosion protection system with both passive resistance and active control and a construction method therefor disclosed in CN202410577461, the system suffers from the problem of protection lag with a response delay of >2 minutes. In high-dynamic corrosion environments such as marine environments, chloride ion penetration or rebar de-passivation may rapidly induce tarnishing, and a system response delay of more than 2 minutes prevents the protective measures from taking effect in time, significantly increasing the risk of early-stage structural damage. Due to the lack of a dynamic threshold determining mechanism, the system relies on fixed thresholds to trigger protection (such as preset potential or current values) and is unable to dynamically adjust a protection strategy according to variables such as a concrete age, ambient temperature and humidity, and a chloride ion diffusion rate. In a cathodic protection system with impressed current for reinforced concrete disclosed in CN115449801A, although the tarnishing time can be predicted by monitoring, the system relies on a fixed proportion of pre-embedded sensors and complex parameter calibration, and a sensor needs to be strictly matched with parameters such as a concrete cover thickness and a chloride ion diffusion coefficient. In practical engineering, fluctuations in concrete mix proportion, differences in construction quality or changes in environmental loads may lead to deviations in monitoring data of the sensor. There are still problems of insufficient adaptability and high maintenance cost in a marine environment or diverse engineering. The system needs to calibrate parameters of the sensor and the chloride ion diffusion model regularly. In an offshore platform or a deep-sea structure far from the mainland, manual calibration is expensive and difficult to achieve real-time performance. For example, in a cathodic protection system for reinforced concrete and a protection method thereof disclosed in CN116445920A, although maintenance requirements are reduced through a combination of a sacrificial anode and an impressed current, the system relies on the layout of an embedded cable trough and a fixed anode, and the cable trough needs to be embedded before concrete pouring, which requires high construction accuracy, and a positioning deviation between the cable trough and an anode hole easily leads to uneven current distribution. A dissolution rate of the sacrificial anode (such as zinc alloy) in seawater immersion or splash zone is accelerated, so that the sacrificial anode needs to be replaced frequently. If an auxiliary anode of an impressed current system (such as Mixed Metal Oxide (MMO) titanium tape) is not fully compacted with conductive mortar, an anode-electrolyte interface is prone to failure due to concrete shrinkage cracks. There are still problems of cumbersome mounting and limited adaptability in a complex marine environment.

For example, in the prior art, a high-toughness conductive composite anode material for cathodic protection of a reinforced concrete structure and a preparation method therefor are disclosed in CN116445920A. Although electrical conductivity is enhanced through carbon fibers, it still needs to rely on fixed proportions and complex dispersion processes. The material components need to be prepared in strict accordance with the preset ratios. In practical engineering, due to differences in concrete strength grades, aggregate particle sizes, or fluctuations in ambient temperature and humidity, the carbon fibers need to be pre-dispersed with a sodium carboxymethylcellulose dispersant, which involves multi-step stirring and impregnating adhesive penetration processes and takes as long as 2-3 hours. This process has extremely high requirements on the cleanliness of a construction environment. If dust pollution or temperature control deviation occurs on site, fiber agglomeration or uneven solidification of colloid may be caused, which seriously affects the uniformity of a protection current, resulting in insufficient stability of a conductive network and difficulty in giving consideration to both sensing accuracy and dynamic response ability. A material design focuses on the optimization of static conductivity, and lacks a sensing and feedback mechanism for dynamic variables such as the chloride ion penetration rate and potential drift of the rebar. In CN117024070A, steel tarnishing-resistant self-sensing concrete and a preparation method therefor are provided. Only a one-way monitoring function can be achieved, chloride ion accumulation or microcrack propagation can be reflected only through changes in resistivity (such as, a threshold value is ≥15%), and it is unable to link a control module to trigger protective measures. A closed-loop control circuit cannot be formed, as a threshold adaptive algorithm and an actuator interface are not incorporated into the system, an output of the protective current needs to be set in advance and cannot be dynamically adjusted according to a real-time potential. In addition, high sensitivity to environmental interference makes a conductive network of the self-sensing concrete susceptible to the effects of temperature and humidity as well as carbonation depth. In CN115370066A, an impact-resistance reinforcement structure for a concrete member with a self-sensing function and a manufacturing method therefor are provided. Although damage monitoring can be achieved an optical fiber cable, this method relies on an external optical fiber protective layer and a complex anchoring mounting process, the optical fiber cable should be wrapped and protected by a large rupture strain-fiber-reinforced polymer (LRS-FRP) strain band. However, an interface between fiber-reinforced polymer (FRP) and the concrete is prone to debonding under an impact load, resulting in fiber exposure and fracture. This involves complicated construction procedures: pre-drilling holes of Φ10 mm×60 mm for mounting anchors (at a spacing of 20-50 cm), as well as achieving precise alignment of connecting holes(with an error margin of <1 mm) , which leads to low construction efficiency.

In conclusion, there are problems in three aspects in the current reinforced concrete corrosion protection technology. (1) Slow response and insufficient dynamic regulation capability. It is impossible to dynamically adjust strategies according to variables such as a concrete age, ambient temperature and humidity, and a chloride ion diffusion rate, which leads to delayed protection in high-dynamic environments such as marine environments and significantly increases the risk of early structural damage. (2) Poor adaptability and high maintenance cost. This technology relies on pre-embedded sensors, cable troughs or fixed-proportion materials, however, in practical engineering, fluctuations in concrete mix proportion, construction error, or environmental interference may lead to deviations in monitoring data, non-uniform current distribution, and expensive maintenance cost. (3) Complex construction and function singularity defect. An existing scheme needs to embed the cable trough or an anchor in advance or requires a complex dispersion process, leading to low construction efficiency and easy failure due to interface debonding. In addition, most technologies only achieve one-way monitoring or static protection, lack closed-loop control links, and cannot adjust pulse currents in a coordinated manner to cope with dynamically corrosive environments.

### SUMMARY

To overcome the deficiencies of existing intelligent concrete, such as single monitoring function and limited load adaptation range due to the limitation of an elastic limit of a matrix, the present disclosure provides a reinforced concrete dynamic protection system with both damage self-sensing and active corrosion. By co-doping copper nanofibers, a sodium polystyrene sulfonate dispersant and a titanate coupling agent, combined with grid-shaped stainless steel electrodes and solar pulsed direct current regulation technology, integration of multi-parameter self-sensing and dynamic protection is achieved, featuring an environmental interference-resistant design, a dynamic threshold control and pulsed protection mechanism, adaptive damage threshold adjustment, pulse direct current protection, optimization of crack resistance and a construction adaptability, as well as innovative fiber synergistic reinforcement. This breaks through the technical bottlenecks of conventional technologies, such as single monitoring function, slow response and complicated construction, and provides a full-life-cycle intelligent protection solution for a reinforced concrete structure under harsh environments including high salt spray and wet-dry alternation.

The technical solution adopted by the present disclosure to solve the technical problem is as follows.

A reinforced concrete dynamic protection system with both damage self-sensing and active corrosion protection includes a rebar skeleton, conductive self-sensing concrete, stainless steel electrodes, a solar power supply, and a monitoring control system, where the rebar skeleton serves as both a cathode and a force-bearing main body of a concrete structure; the conductive self-sensing concrete is configured for wrapping the rebar skeleton and is composed of a cement-based material, copper nanofibers, and sodium polystyrene sulfonate dispersant; the stainless steel electrodes are distributed in a grid pattern and are fixedly connected to the conductive self-sensing concrete;
the rebar skeleton is connected to a negative electrode of the solar power supply, and the conductive self-sensing concrete is poured around the rebar skeleton as a conductive path and crack monitoring; the stainless steel electrode is buried on a surface layer of the self-sensing concrete and is connected to an anode of the solar power supply to form a cathodic protection system loop; and the monitoring control system is configured to analyze an electrical signal in the system loop and control a frequency and voltage of a pulsed current.

Further, a spacing between the stainless steel electrodes in the grid pattern is 20-50 cm, and the stainless steel electrodes are fixed to the conductive self-sensing concrete by an epoxy resin adhesive layer.

Still further, a damage threshold of the monitoring control system is dynamically adjusted according to an age of the conductive self-sensing concrete, when the age is ≤7 days, the threshold is set as a sudden change in resistivity of ≥15%; and when the age is >7 days, the threshold is set as a sudden change in resistivity of ≥20%.

In an embodiment, the solar power supply includes a photovoltaic panel, an energy storage battery, and a voltage regulator; and an output voltage ranges from 1.5 V to 5 V, and a current density ranges from 10m A/m² to 50 mA/m².

In the cathodic protection system loop, an application mode of a cathodic protection voltage is pulsed direct current, where a pulse frequency is 1-10 Hz and a duty cycle is 20%-50%.

The conductive self-sensing concrete is prepared from the following components in mass percentage:
95.2%-99.35% cement-based material;
0.5%-3% copper nanofibers; and
0.1%-1.5% sodium polystyrene sulfonate dispersant.

The cement-based material is prepared from the following components in mass percentage: 45%-55% 52.5 ordinary Portland cement, 5%-12% silica fume, and 35%-45% quartz sand, where a concrete gradation of the quartz sand is 20-100 mesh.

The copper nanofibers have a diameter of 50-200 nm and a length of 1-5 μm, which are uniformly dispersed by the sodium polystyrene sulfonate dispersant and coated with a silicon dioxide insulating layer on their surfaces, where the silicon dioxide insulating layer has a thickness of 10-50 nm, and a breakdown field strength of ≥20 kV/mm.

In an embodiment, the conductive self-sensing concrete is doped with a titanate coupling agent in a mass proportion of 0.05%-0.3%, which is used for enhancing an interfacial bonding force between the copper nanofibers and a cement matrix as well as electrical conductivity.

The technical concept of the present disclosure is as follows. Through a copper nanofiber conductive network design, the concrete is endowed with following advantages. 1) High electrical conductivity and stability: the copper nanofibers (with a diameter of 50-200 nm and a length of 1-5 μm) are synthesized by an electrochemical deposition method and coated with a silicon dioxide insulating layer (with a thickness of 10-50 nm, and a breakdown field strength of ≥20 kV/mm) on surfaces of the copper nanofibers, thereby forming a three-dimensional conductive network in a concrete matrix. The silicon dioxide layer can effectively isolate oxidation corrosion of the copper fibers caused by an alkaline environment of the concrete and ensure long-term electrical conductivity stability (the 28-day resistivity drift is <3%). 2) Dispersion and interface optimization: the sodium polystyrene sulfonate dispersant (0.1%-1.5% by mass) is combined with the titanate coupling agent (0.05%-0.3% by mass) to achieve uniform dispersion of fibers and enhance a fiber-matrix interfacial bonding force, with a compressive strength loss rate of ≤5%. In addition, the continuity of a conductive network during microcrack propagation is improved (a resistivity fluctuation is <5% when a crack width is less than <0.3 mm).

Through a dynamic anti-corrosion regulation mechanism, the system is endowed with following advantages. 1) An intelligent threshold response: the monitoring system can dynamically adjust a damage threshold according to an age of the concrete (when the age is ≤7 days, a sudden change in resistivity is ≥15%, and when the age is >7 days, the sudden change in resistivity is ≥20%), and collect the resistivity, temperature and humidity data in real time for accurately determining a damage level. 2) Pulsed direct current protection: the solar power supply (with an output voltage of 1.5-5 V and a current density of 10-50 mA/m²) applies a pulsed voltage (with a frequency of 1-10 Hz and a duty cycle of 20%-50%) through stainless steel electrodes in a grid pattern (with a spacing of 20-50 cm), and a cathodic protection potential is regulated to a range of −720 to −1100 mV(vs Ag/AgCl), thereby inhibiting chloride ion penetration and repairing a passivation film and increasing the protection efficiency by more than 40%.

Through synergistic effect of the material and system, the system is endowed with following advantages. 1) Electrode layout optimization: the stainless steel electrodes (with a size of 35 mm×60 mm, and meshes of 4-8) are fixed to the surface of the concrete through an epoxy resin adhesive layer, a construction spacing error is allowed to be ±5 cm, thereby adapting to the curved surface or irregular structures and ensuring current distribution uniformity. 2) Environment adaptability: the system can operate stably in a temperature range of −20° C. to 80° C. and dry to fully saturated moisture conditions, the resistivity sensitivity reaches ±0.5%, and a humidity monitoring error is <3%.

By material innovation and system integration, the present disclosure solves the problems of single function, slow response and complicated construction in the conventional intelligent concrete, thereby providing a full-life-cycle and high-reliability protection solution for the reinforced concrete structure under harsh working conditions such as marine environment and bridges.

The present disclosure has beneficial effects as follows.
(1) Resistivity sensitivity: the damage threshold is dynamically adjusted according to the age (when the age is ≤7 days, the sudden change in resistivity is ≥15%, and when the age is >7 days, the sudden change in resistivity is ≥20%).
(2) Environment adaptability: at a low temperature of −20° C. or a high temperature of 80° C., a resistivity fluctuation is <8%, and a humidity monitoring error is <3%.
(3) Damage resistance and durability: the insulating layer made of copper nanofibers can resist corrosion of an alkaline environment, achieving 28-day conductivity stability (the resistivity drift is <3%).
(4) The titanate coupling agent can enhance the interface bonding, the compressive strength loss rate is ≤5%, and an ultimate tensile strain reaches 0.5%.

### DETAILED DESCRIPTION OF THE EMBODIMENTS

The present disclosure is further described below.

A reinforced concrete dynamic protection system with both damage self-sensing and active corrosion protection includes a rebar skeleton, conductive self-sensing concrete, stainless steel electrodes, a solar power supply, and a monitoring control system. The rebar skeleton serves as both a force-bearing main body of a concrete structure and a cathode. The conductive self-sensing concrete is used for wrapping the rebar skeleton and is composed of a cement-based material, copper nanofibers, and sodium polystyrene sulfonate dispersant. The stainless steel electrodes are distributed in a grid pattern and are fixedly connected to the conductive self-sensing concrete.

The rebar skeleton is connected to a negative electrode of the solar power supply, and the conductive self-sensing concrete is poured around the rebar skeleton as a conductive path and crack monitoring. The stainless steel electrode is buried on a surface layer of the self-sensing concrete and is connected to an anode of the solar power supply to form a cathodic protection system loop. The monitoring control system is configured to analyze an electrical signal in the system loop and control a frequency and voltage of a pulsed current.

Further, a spacing between the stainless steel electrodes in the grid pattern is 20-50 cm, and the electrodes are fixed to the conductive self-sensing concrete by an epoxy resin adhesive layer.

Still further, a damage threshold of the monitoring control system is dynamically adjusted according to an age of the concrete, when the age is ≤7 days, the threshold is set as a sudden change in resistivity of ≥15%; and when the age is >7 days, the threshold is set as a sudden change in resistivity of ≥20%.

In an embodiment, the solar power supply includes a photovoltaic panel, an energy storage battery, and a voltage regulator; and an output voltage ranges from 1.5 V to 5 V, and a current density ranges from 10m A/m² to 50 mA/m².

In the cathodic protection system loop, an application mode of a cathodic protection voltage is pulsed direct current, where a pulse frequency is 1-10 Hz and a duty cycle is 20%-50%.

The conductive self-sensing concrete is prepared from the following components in mass percentage:
95.2%-99.35% cemented-based material;
0.5%-3% copper nanofibers; and
0.1%-1.5% sodium polystyrene sulfonate dispersant.

The cement-based material is prepared from the following components in mass percentage: 45%-55% 52.5 ordinary Portland cement, 5%-12% silica fume, and 35%-45% quartz sand, where a concrete gradation of the quartz sand is 20-100 mesh.

The copper nanofibers have a diameter of 50-200 nm and a length of 1-5 μm, which are uniformly dispersed by the sodium polystyrene sulfonate dispersant and coated with a silicon dioxide insulating layer on the their surfaces, where the silicon dioxide insulating layer has a thickness of 10-50 nm, and a breakdown field strength of ≥20 kV/mm.

In an embodiment, the conductive self-sensing concrete is doped with a titanate coupling agent in a mass proportion of 0.05%-0.3%, which is used for enhancing an interfacial bonding force between the copper nanofibers and a cement matrix as well as electrical conductivity.

A cracking sensing protection system based on self-sensing concrete in this embodiment includes conductive self-sensing concrete and dynamic protection system.

Conductive self-sensing concrete includes a matrix and functional phase.

The matrix is prepared from the following materials: 52.5 ordinary Portland cement (45%-55%), silica fume (5%-12%), and quartz sand (35%-45%, and a concrete gradation of the quartz sand is 20-100 mesh).

Functional phase includes the copper nanofibers and the Titanate coupling agent.

The copper nanofibers have a diameter of 50-200 nm and a length of 1-5 μm, which are uniformly dispersed by the sodium polystyrene sulfonate dispersant (0.1%-1.5%) and coated with a silicon dioxide insulating layer (with a thickness of 10-50 nm, and a breakdown field strength of ≥20 kV/mm) on their surfaces.

A doping amount of the titanate coupling agent is 0.05%-0.3%, so that a fiber-matrix interfacial bonding force can be improved, and a 28-day loss rate of compressive strength is ≤5%.

Dynamic protection system includes electrodes and solar energy regulation module.

Electrode layout: the stainless steel electrodes (with a spacing of 20-50 cm, a size of 35 mm×60 mm, and meshes of 4-8) in grid pattern are fixed to the surface of the concrete through an epoxy resin adhesive layer to form uniform current distribution.

In solar energy regulation module, a photovoltaic panel outputs a pulsed direct current (at a voltage of 1.5-5 V and a current density of 10-50 mA/m²), the frequency is 1-10 Hz, and the duty cycle is 20%-50%, and a cathode potential is regulated to a range from −720 mV to −1100 mV (vs Ag/AgCl) in real time.

A preparation process of a cracking sensing protection system based on self-sensing concrete in this embodiment includes copper nanofiber pretreatment and concrete blending and construction.

In copper nanofiber pretreatment, a copper salt solution (0.1-0.5 mol/L) is mixed with an ascorbic acid reducing agent, and synthesized into fibers by an electrochemical deposition method, followed by impregnation with silica sol and high-temperature curing.

Concrete blending and construction includes dry material mixing, slurry preparation and casting and forming.

In dry material mixing, cement, silicon fume, quartz sand and a water reducing admixture (0.02%-0.03%) are stirred at a low speed for 3-5 min.

In slurry preparation, copper nanofiber dispersion liquid and a titanate coupling agent are added in turn and undergo high-speed shear mixing (8000-12000 r/min, 5-8 min) until the materials are uniformly mixed.

In casting and forming, the mixture is injected into a mold, an electrode is into the mixture for vibrating for 20-40 seconds, followed by standard curing (referring to curing at 20° C. and relative humidity above 95%) until 28 days.

The scheme of this embodiment breaks through the limitation of single function in the conventional technology, real-time electrical signal feedback of load/damage is achieved via conductive self-sensing concrete (composed of cement-based matrix, nano-conductive fibers and conductive polymer dispersant). In addition, an in-situ electrochemical system with a rebar as a cathode and self-sensing concrete as an anode is constructed to form a “sensing-determining-response” closed loop, thus achieving the first deep integration of structural health monitoring and active anti-corrosion technology.

High-performance material synergistic interaction: the compounded use of the nano conductive fibers and a conductive polymer dispersant significantly enhance the stability of a concrete conductive network. A three-dimensional conductive skeleton constructed by the nanofibers can enhance the mechanical-electrical coupling sensitivity, and the conductive polymer optimizes the transfer efficiency of interfacial charges, so that the accuracy of a damage sensing threshold can be improved by 80% and more, and the fluctuation in material resistivity is reduced.

Solar energy-driven intelligent protection: a solar power supply is linked with a monitoring control system, and active anti-corrosion can be triggered only when the concrete damage exceeds a preset threshold, so that the energy consumption is reduced by over 70% in comparison with conventional potentiostatic cathodic protection technology. The system has adaptive adjustment capability and can dynamically optimize the output current according to a crack width of concrete cracking, thereby prolonging the service life of the rebar structure.

Full life-cycle cost optimization: more than 60% of structural repair cost can be reduced through early damage warning, and an active anti-corrosion system can reduce a rebar corrosion rate by 90%, significantly prolonging the service life of the structure. A built-in electrode design eliminates the mounting and maintenance cost of external auxiliary electrodes, and a full-system modular design facilitates the renovation and upgrading of an existing structure.

By introducing a dynamic threshold adaptive algorithm, an integrated design of self-sensing concrete (where a conductive network is constructed with copper nanofibers and titanate coupling agent) and a modular electrode layout, the solution of this embodiment abandons the limitations of pre-embedded sensors and fixed-proportion materials, and can achieve millisecond-level response, full-life-cycle protection and complex environment self-adaptation by combining solar pulsed direct current regulation (1.5-5 V, 10-50 mA/m²). This breaks through the technical bottlenecks of conventional technologies such as slow response, cumbersome construction and single function.

The contents described in embodiments of this specification are merely enumerations of the implementation forms of the inventive concept and are provided for illustrative purposes only. The scope of protection of the present disclosure shall not be considered to be limited to the specific forms set forth in the embodiment. The scope of protection of the present disclosure shall also cover equivalent technical means that those of ordinary skill in the art can conceive in accordance with the concept of the present disclosure.

What is claimed is:

1. A reinforced concrete dynamic protection system with both damage self-sensing and active corrosion protection, comprising a rebar skeleton, conductive self-sensing concrete, stainless steel electrodes, a solar power supply, and a monitoring control system, wherein the rebar skeleton serves as both a force-bearing main body of a concrete structure and a cathode; the conductive self-sensing concrete is configured for wrapping the rebar skeleton and is composed of a cement-based material, copper nanofibers, and sodium polystyrene sulfonate dispersant; the stainless steel electrodes are distributed in a grid pattern and are fixedly connected to the conductive self-sensing concrete;
the rebar skeleton is connected to a negative electrode of the solar power supply, and the conductive self-sensing concrete is poured around the rebar skeleton as a conductive path and crack monitoring; the stainless steel electrode is buried on a surface layer of the self-sensing concrete and is connected to an anode of the solar power supply to form a cathodic protection system loop; and the monitoring control system is configured to analyze an electrical signal in the cathodic protection system loop and control a frequency and voltage of a pulsed current.

2. The reinforced concrete dynamic protection system with both damage self-sensing and active corrosion protection according to claim 1, wherein a spacing between the stainless steel electrodes in the grid pattern is 20-50 cm, and the stainless steel electrodes are fixed to the conductive self-sensing concrete by an epoxy resin adhesive layer.

3. The reinforced concrete dynamic protection system with both damage self-sensing and active corrosion protection according to claim 1, wherein a damage threshold of the monitoring control system is dynamically adjusted according to an age of the conductive self-sensing concrete, when the age is ≤7 days, the threshold is set as a sudden change in resistivity of ≥15%; and when the age is >7 days, the threshold is set as the sudden change in the resistivity of ≥20%.

4. The reinforced concrete dynamic protection system with both damage self-sensing and active corrosion protection according to claim 2, wherein a damage threshold of the monitoring control system is dynamically adjusted according to an age of the conductive self-sensing concrete, when the age is ≤7 days, the threshold is set as a sudden change in resistivity of ≥15%; and when the age is >7 days, the threshold is set as the sudden change in the resistivity of ≥20%.

5. The reinforced concrete dynamic protection system with both damage self-sensing and active corrosion protection according to claim 1, wherein the solar power supply comprises a photovoltaic panel, an energy storage battery, and a voltage regulator; and an output voltage ranges from 1.5 V to 5 V, and a current density ranges from 10m A/m² to 50 mA/m².

6. The reinforced concrete dynamic protection system with both damage self-sensing and active corrosion protection according to claim 2, wherein the solar power supply comprises a photovoltaic panel, an energy storage battery, and a voltage regulator; and an output voltage ranges from 1.5 V to 5 V, and a current density ranges from 10m A/m² to 50 mA/m².

7. The reinforced concrete dynamic protection system with both damage self-sensing and active corrosion protection according to claim 1, wherein in the cathodic protection system loop, an application mode of a cathodic protection voltage is pulsed direct current, wherein a pulse frequency is 1-10 Hz and a duty cycle is 20%-50%.

8. The reinforced concrete dynamic protection system with both damage self-sensing and active corrosion protection according to claim 2, wherein in the cathodic protection system loop, an application mode of a cathodic protection voltage is pulsed direct current, wherein a pulse frequency is 1-10 Hz and a duty cycle is 20%-50%.

9. The reinforced concrete dynamic protection system with both damage self-sensing and active corrosion protection according to claim 1, wherein the conductive self-sensing concrete is prepared from following components in mass percentage:
95.2%-99.35% cemented-based material;
0.5%-3% copper nanofibers; and
0.1%-1.5% sodium polystyrene sulfonate dispersant.

10. The reinforced concrete dynamic protection system with both damage self-sensing and active corrosion protection according to claim 2, wherein the conductive self-sensing concrete is prepared from following components in mass percentage:
95. 2%-99.35% cemented-based material;
0.5%-3% copper nanofibers; and
0.1%-1.5% sodium polystyrene sulfonate dispersant.

11. The reinforced concrete dynamic protection system with both damage self-sensing and active corrosion protection according to claim 9, wherein the cement-based material is prepared from following components in mass percentage: 45%-55% 52.5 ordinary Portland cement, 5%-12% silica fume, and 35%-45% quartz sand, wherein a concrete gradation of the quartz sand is 20-100 mesh.

12. The reinforced concrete dynamic protection system with both damage self-sensing and active corrosion protection according to claim 10, wherein the cement-based material is prepared from following components in mass percentage: 45%-55% 52.5 ordinary Portland cement, 5%-12% silica fume, and 35%-45% quartz sand, wherein a concrete gradation of the quartz sand is 20-100 mesh.

13. The reinforced concrete dynamic protection system with both damage self-sensing and active corrosion protection according to claim 9, wherein the copper nanofibers have a diameter of 50-200 nm and a length of 1-5 μm, which are uniformly dispersed by the sodium polystyrene sulfonate dispersant and coated with a silicon dioxide insulating layer on their surfaces, wherein the silicon dioxide insulating layer has a thickness of 10-50 nm, and a breakdown field strength of ≥20 kV/mm.

14. The reinforced concrete dynamic protection system with both damage self-sensing and active corrosion protection according to claim 10, wherein the copper nanofibers have a diameter of 50-200 nm and a length of 1-5 μm, which are uniformly dispersed by the sodium polystyrene sulfonate dispersant and coated with a silicon dioxide insulating layer on their surfaces, wherein the silicon dioxide insulating layer has a thickness of 10-50 nm, and a breakdown field strength of ≥20 kV/mm.

15. The reinforced concrete dynamic protection system with both damage self-sensing and active corrosion protection according to claim 9, wherein the conductive self-sensing concrete is doped with a titanate coupling agent in a mass proportion of 0.05%-0.3%, which is used for enhancing an interfacial bonding force between the copper nanofibers and a cement matrix as well as electrical conductivity.

16. The reinforced concrete dynamic protection system with both damage self-sensing and active corrosion protection according to claim 10, wherein the conductive self-sensing concrete is doped with a titanate coupling agent in a mass proportion of 0.05%-0.3%, which is used for enhancing an interfacial bonding force between the copper nanofibers and a cement matrix as well as electrical conductivity.
